(19) United States
(12) Patent Application Publication
(10) Pub. No.: US 2024/0165086 A1
(43) Pub. Date: May 23, 2024

(54) COATED FAMOTIDINE PARTICLE

(71) Applicant: Johnson & Johnson Consumer Inc., Skillman, NJ (US)

(72) Inventor: Anurag PANDEY, Lower Gwynedd, PA (US)

(21) Appl. No.: 18/552,211

(22) PCT Filed: Mar. 9, 2022
(86) PCT No.: PCT/IB2022/052110

§ 371 Date: Sep. 25, 2023

Related U.S. Application Data
(60) Provisional application No. 63/165,198, filed on Mar. 24, 2021.

(30) Foreign Application Priority Data
Apr. 6, 2021 (SE) 2150425-3

(51) Int. Cl. A61K 31/426; A61J 3/00; A61K 9/16; A61K 9/20; A61K 33/08; A61K 33/10

(52) U.S. Cl. CPC A61K 31/426; A61J 3/005; A61K 9/1611; A61K 9/1623; A61K 9/1652; A61K 9/1682; A61K 9/2013; A61K 9/2018; A61K 9/2027; A61K 9/2081; A61K 33/08; A61K 33/10

(57) ABSTRACT

The invention relates to a coated famotidine particle having at least a core and a coating layer, wherein the particle core comprises famotidine, a first filler and a first binder; and wherein the coating layer is substantially free from famotidine and comprises a second filler and a second binder. The invention also relates to solid dosage forms comprising said coated famotidine particle, and the use of the coated famotidine particle for treating a subject suffering from a disease or disorder in the gastrointestinal tract.

### FIELD OF INVENTION

The invention relates to a coated famotidine particle having at least a core and a coating layer, wherein the particle core comprises famotidine, a first filler and a first binder; and wherein the coating layer is substantially free from famotidine and comprises a second filler and a second binder. The invention also relates to a solid dosage form comprising said coated famotidine particle, and the use of the coated famotidine particle for treating a subject suffering from a disease or disorder in the gastrointestinal tract.

### BACKGROUND OF INVENTION

Histamine H2-receptor antagonists, for example cimetidine, ranitidine, nizetidine, roxatine and famotidine, reduce acid secretion by acting directly on the acid-secreting parietal cell located within the gastric gland of the stomach wall.

Histamine H2-receptor antagonists are of potential benefit in the self-medication of acute, self-limiting gastric disorders such as hyperacidity. However, their slow onset of action is unlikely to meet the consumer requirement for rapid relief of symptoms.

Co-administration of histamine H2-receptor antagonists and other pharmaceutically active materials, including antacids, has been investigated. The rationale for co-administration with antacid is that the antacid brings about rapid relief from the symptoms of excess stomach acidity by neutralization whereas the histamine H2-receptor antagonist acts independently by inhibiting secretion of acid from the parietal cell.

Antacids used today are made from a variety of inorganic salts such as calcium carbonate, sodium bicarbonate, magnesium salts and aluminum salts. Magnesium hydroxide and aluminum hydroxide are the most potent magnesium and aluminum salts and are often used in combination. In addition, aluminum oxide, magnesium oxide, magnesium carbonate, aluminum phosphate, magaldrate, magnesium trisilicate, and aluminum sucrose sulfate (sucralfate) are also employed.

Histamine H2-receptor antagonists such as Famotidine are bitter in taste. To be more appealing to the user Famotidine, for example, requires effective taste-masking before it can be incorporated into oral dosage forms which disintegrate/disperse in the buccal cavity.

Coated Famotidine granules, with taste masking, for the treatment of gastric disorders have already been reported:

U.S. Pat. No. 5,817,340A discloses a solid oral dosage form for the treatment of gastrointestinal disorders comprising a therapeutically effective amount of guanidinothiazole compound; and a therapeutically effective amount of an antacid wherein the pharmaceutical and an antacid are separated by a barrier which is substantially impermeable to an antacid. WO2017091166 and EP0538034 discloses coated famotidine particles, but are silent about the different filles and binders, which provides the new improved properties.

We are now disclosing a new type of Famotidine coated particles with improved properties.

### SUMMARY OF THE INVENTION

The invention relates to the development of new coated famotidine particle having at least a core and a coating layer, wherein
the particle core comprises from 5% to 20% w/w of famotidine, from 70% to 93% w/w of a first filler and from 2% to 10% w/w of a first binder, and
the coating layer is substantially free from famotidine and comprises a second filler and a second binder,
   wherein the first and second filler may be similar or different; and the first and second binder may be similar or different,
   % w/w with respect to the particle core total weight.

The invention relates to a solid dosage form comprising the new coated famotidine particle according to the invention, wherein the coated famotidine particle represents from 3% to 6% w/w of the solid dosage form total weight.

The invention also relates to a method for manufacturing the new coated famotidine particle according to the invention, comprising the following steps:
Spraying a first binder on a mixture of famotidine and a granulation batch of a first filler to obtain wet famotidine particles core,
Spraying a second binder, while mixing said wet famotidine particles core and a layering batch comprising a second filler, to coat the famotidine particle core into a coated famotidine particle,
   wherein the first and second filler may be similar or different; and the first and second binder may be similar or different.

Finally, the invention relates to a method of using the new coated famotidine particle and the solid dosage form comprising the new coated famotidine particle according to the invention, for the treatment of a subject suffering from a disease or disorder in the gastro intestinal tract, such as heart burn.

### DETAILED DESCRIPTION OF EMBODIMENTS OF THE INVENTION

### Definitions

In the context of the present application and invention the following definitions apply:

A “dosage”, “dosage form”, “dose unit” or “dose” as used herein means the amount of a pharmaceutical ingredient comprising therapeutically active agent(s) administered at a time. “Dosage”, “dosage form”, “dose unit” or “dose” includes administration of one or more units of pharmaceutical ingredient administered at the same time.

The term substantially free from famotidine is intended to mean that the coating layer contains less than 5% w/w of Famotidine, preferably less than 1% w/w, more preferably less than 0.5% w/w or even less than 0.1% w/w of Famotidine (% w/w with respect to the coating layer total weight).

The term “gastric disease or disorder” is primarily intended to mean an increased production of the acid secretion which leads to heartburn and bothersome gas symptoms in a subject, also named indigestion. Indigestion, also known as dyspepsia, is a condition of impaired digestion. Symptoms may include upper abdominal fullness, heartburn, nausea, belching, or upper abdominal pain. People may also experience feeling full earlier than expected when eating. Dyspepsia is a common problem and is frequently caused by gastroesophageal reflux disease (GERD) or gastritis.

According to the present invention a binder is defined as a material used to adhere particles to other particles and facilitate agglomeration, typically through the addition of water as a sprayed solution or within a granulation. Typically these binders have a high degree of surface wetting and spreadability and a high degree of wet adhesion (strong liquid bridges in the wet granules) to allow the formation of agglomerates, while also possessing plasticity in the dry state to overcome unfavorable powder flow and mechanical properties.

According to the present invention a filler is defined as a pharmaceutically inert, water soluble or insoluble powder. Fillers are added to the tablet formulation and act as a diluent. Fillers bulk up tablets containing potent drug substance often in very small quantities. Fillers may also help improve powder flow characteristic and tableting.

In one embodiment the invention relates to a coated famotidine particle having at least a core and a coating layer, wherein
the particle core comprises from 5% to 20% w/w of famotidine, from 70% to 93% w/w of a first filler and from 2% to 10% w/w of a second binder, and
the coating layer is substantially free from famotidine and comprises a second filler and a second binder,
   wherein the first and second filler may be similar or different; and the first and second binder may be similar or different,
   % w/w with respect to the particle core total weight.

Preferably the first filler and the second filler may be similar.

Preferably the first binder and the second binder may be similar.

Preferably the first filler and second filler may be similar, and the first binder and the second binder may be similar.

The weight percentage of Famotidine in the particle core may be selected from: 5% to 15% w/w, 10% to 20% w/w, 10% to 15% w/w, 5% to 10% w/w, preferably 12% to 14% w/w (% w/w with respect to the particle core total weight).

The weight percentage of first filler in the particle core may be selected from: 70% to 85% w/w, 75% to 85% w/w, 70% to 82% w/w, 75% to 90% w/w, 75% to 82% w/w, 78% to 90% w/w, 78% to 85% w/w, preferably 78% to 82% w/w (% w/w with respect to the particle core total weight).

The weight percentage of first Binder in the particle core may be selected from: 2% to 7% w/w, 5% to 10% w/w, 5% to 8% w/w, preferably 5% to 7% w/w (% w/w with respect to the particle core total weight).

In one example of the coated famotidine particle the first binder and second binder may be selected from the group consisting of hypromellose (hydroxypropylmethylcellulose or HPMC), hydroxypropylcellulose (HPC), methylcellulose, polyvinylpyrrolidone, Sodium Carboxymethylcellulose, Ethyl cellulose, Copovidone (polyvinyl alcohol-povidone copolymer), pregelatinized starch, or combination thereof. The first binder functions to allow famotidine particles to adhere to one other, contributing to particle growth.

In another example of the coated famotidine particle the first filler and second filler may be selected from the group consisting of lactose, microcrystalline cellulose, starch, dextrose, mannitol, sorbitol, xylitol, maltitol or combination thereof. The first filler contributes as a bulking agent for the particle during the particle growth phase of famotidine particles with the addition of the first binder.

In a preferred embodiment of the coated famotidine particle, the first binder and second binder is Hydroxypropylmethylcellulose (or HMPC) and the first filler and second filler is Lactose or a derivative thereof. The second binder allows for the second filler to adhere to the particles, creating a taste-masking layer that does not contain famotidine. This taste-masking layer is comprised of the second binder and second filler.

Optionally the coated famotidine particle core may comprise a flowing agent selected from the group consisting of Silica, colloidal silica, fumed silica, aluminometasilicate, preferably a colloidal silica such as amorphous silicon dioxide.

The weight percentage of flowing agent in the particle core may be selected from: 0.05% to 3% w/w, 0.1% to 2.5% w/w, 0.1% to 2% w/w, 0.1% to 1.5% w/w, 0.1% to 1% w/w, preferably 0.4% to 0.6% w/w (% w/w with respect to the particle core total weight).

The coated famotidine particle according to the invention may have a coating layer accounting for 10% to 30% w/w of the coated famotidine particle total weight.

The coating layer may comprise more than 90% w/w, or more than 95% w/w, preferably more than 97% w/w of a second filler (% w/w with respect to the coating layer total weight). The second filler present in the coating layer may be the same filler as the first filler in the particle core.

The coating layer may comprise less than 5% w/w, preferably less than 2% w/w of a second binder (% w/w with respect to the coating layer total weight). The second binder present in the coating layer may be the same binder as the first binder in the particle core.

The coating layer may comprise less than 2% w/w, preferably less than 1.5% w/w of flowing agent (% w/w with respect to the coating layer total weight). The flowing agent present in the coating layer may be the same flowing agent as in the particle core.

For example, the coating layer may have a thickness comprised between 50 μm to 300 μm, preferably between 100 μm to 200 μm.

In an embodiment the coated famotidine particle may comprise in the particle core and the coating layer:
from 10% to 15% w/w of famotidine, preferably from 12% to 14% w/w,
from 3% to 10% w/w of binders (first and second binders), preferably from 5% to 6.5% w/w,
from 75% to 90% w/w of fillers (first and second fillers), preferably from 80% to 83% w/W,
from 0.1% to 1% w/w of a flowing agent, preferably from 0.5% to 0.7% w/W,
   wherein all % w/w are respective to the coated famotidine particle total weight.

The coated famotidine particle of the invention may have a particle size comprised between 200 μm to 500 μm, preferably between 300 μm to 350 μm.

In one embodiment, the invention relates to a solid dosage form comprising the coated famotidine particle as disclosed above, wherein the coated famotidine particle may represent from 3% to 6% w/w of the solid dosage form total weight.

Preferably the solid dosage form according to the invention may remain in the oral cavity for an amount of time sufficient to allow a partial disintegration, either by chewing or by disintegration with the saliva. Preferably, the solid dosage form may not be swallowed directly after being placed in the oral cavity.

Appropriate solid dosage form may be selected from the group consisting of chewable tablets, orally disintegrating tablets, thin films, lozenges, soft chewables, gummies.

Preferably the solid dosage form according to the invention may be a chewable tablet or an orally disintegrating tablet.

The term “soft chewable” is intended to mean a dosage form which retains its integrity and texture upon chewing, does not break into discrete, solid pieces or particulates upon chewing and is intended to be swallowed. The soft chew is palatable, edible, and is similar in texture to confectionery taffy or nougat.

Preferably the solid dosage form may comprise an antacid in addition to the coated famotidine particle, wherein the antacid may represent from 50% to 60% w/w of the dosage form total weight.

In one example, the solid dosage form contains an antacid that may be selected from the group consisting of calcium carbonate, sodium bicarbonate, magnesium hydroxide, aluminum oxide, aluminum hydroxide, magnesium oxide, magnesium carbonate, aluminum phosphate, magaldrate, magnesium trisilicate, bismuth salicylate, bismuth subsalicylate or combination thereof. Preferably the solid dosage form contains calcium carbonate and magnesium hydroxide as antiacid.

Preferably the antiacid is located in an additional layer to the solid dosage form.

The solid dosage form may have a famotidine content from 50 mg to 200 mg, preferably from 70 mg to 150 mg.

The solid dosage form may further comprise one or more ingredient(s) selected from the list consisting of, colorings, flavors, sweeteners, antioxidants, preservatives, lubricants, glidants, and disintegrants.

Example of flavors are peppermint, spearmint, eucalyptus, licorice, vanilla, caramel, mixed berries, mixed fruits, black current, blue berry, cherry and lemon.

Suitable lubricants include long chain fatty acids and their salts, such as magnesium stearate and stearic acid, talc, glycerides waxes, and mixtures thereof. Suitable glidants include colloidal silicon dioxide.

Examples of sweeteners include, synthetic or natural sugars; artificial sweeteners such as saccharin, sodium saccharin, sucralose, aspartame, acesulfame, thaumatin, glycyrrhizin, sucralose, cyclamate, dihydrochalcone, alitame, miraculin and monellin; sugar alcohols such as sorbitol, mannitol, glycerol, lactitol, maltitol, and xylitol; sugars extracted from sugar cane and sugar beet (sucrose), dextrose (also called glucose), fructose (also called laevulose), and lactose (also called milk sugar); isomalt, stevia, and mixtures thereof.

Examples of coloring agents include lakes and dyes approved as a food additive.

In another embodiment, the invention relates to a method for manufacturing a coated famotidine particle as disclosed above, comprising the following steps:
Spraying a first binder on a mixture of famotidine and a granulation batch of a first filler to obtain wet famotidine particles core,
Spraying a second binder, while mixing said wet famotidine particles core and a layering batch comprising a second filler, to coat the famotidine particle core into a coated famotidine particle,
   wherein the first and second filler may be similar or different; and the first and second binder may be similar or different.

Optionally, the method may comprise an additional step of spraying the second binder onto the coated famotidine particle to seal the coated particle.

The spraying of a first binder on a mixture of famotidine and a granulation batch of a first filler to obtain wet famotidine particles core, may be a granulation step.

The spraying of a second binder, while mixing said wet famotidine particles core and a layering batch comprising a second filler may be a layering or coating step.

The spraying of the second binder onto the coated famotidine particle may be a sealing step.

The method may also encompass the situation where the first filler and the second filler used respectively in the granulation batch and the layering batch are the same. For example, the filler may be lactose or derivative thereof, for both steps.

The method may also encompass the situation where the first binder and the second binder, respectively sprayed on the famotidine and the granulation batch, and sprayed on the wet famotidine particles core and the layering batch, are the same. For example, the binder may be Hydroxypropylmethylcellulose for both steps.

Preferably the method may not require the use of organic solvent in any of the steps.

In another embodiment the invention relates to the use of a coated famotidine particle as described above, for the manufacture of a solid dosage form for the treatment of a disease or disorder in the gastro intestinal tract such as heartburn, bothersome gas symptoms, indigestion, dyspepsia, impaired digestion, upper abdominal fullness, nausea, belching, upper abdominal pain, gastroesophageal reflux disease (GERD) or gastritis.

In other words, the coated famotidine particle as described above may be used as a treatment of a disease or disorder in the gastro intestinal tract such as heartburn, heartburn, bothersome gas symptoms, indigestion, dyspepsia, impaired digestion, upper abdominal fullness, nausea, belching, upper abdominal pain, gastroesophageal reflux disease (GERD) or gastritis.

The solid dosage as described above may be used as a treatment of a disease or disorder in the gastro intestinal tract such as heartburn, heartburn, bothersome gas symptoms, indigestion, dyspepsia, impaired digestion, upper abdominal fullness, nausea, belching, upper abdominal pain, gastroesophageal reflux disease (GERD) or gastritis.

Finally the invention may relate to a method of treating a disease or disorder in the gastro intestinal tract such as heartburn, heartburn, bothersome gas symptoms, indigestion, dyspepsia, impaired digestion, upper abdominal fullness, nausea, belching, upper abdominal pain, gastroesophageal reflux disease (GERD) or gastritis; by use of the coated famotidine particle as described above, or the solid dosage form as described above.

### EXAMPLES

### Example 1: Granulation Step Using Huttlin Fluid Bed Coating Unit

Part A: HPMC Solution Preparation
1. 1200 g of purified water was added to a 2 L container and heated to 70ºC.
2. 150 g of Hypromellose (HPMC) ESLV was added slowly while mixing and the heat was turned off.
3. The solution was allowed to cool while mixing and purified water was added to the solution up to a solution total mass of 1500 g to obtain a 10% weight/weight solution.

Part B: Granulation Step

Powder Blend for Granulation
1. 1610 g of Lactose monohydrate impalpable NF, 300 g of Famotidine HCl, and 10 g Colloidal Silica (Syloid 244FP) was weighed into a bag-lined tared container.
2. The mixture was manually blended in the bag as the Granulation Blend (as shown in Table 1, similar for trials 1, 2 and 3).

Powder Blend for Layering (Trials 1 and 2)
1. 400 g of Lactose monohydrate impalpable NF, 6 g of HPMC E5 LV, and 4 g Syloid 244FP was weighed into a bag lined tared container.
2. The mixture was manually blended in the bag as the Layering Powder (as shown in Table 2a, for trials 1 and 2).
   An alternative powder blend for layering is disclosed in Table 2b (trial 3).

Granulation and Layering: (Process Parameters shown in Table 3)
1. 1920 g of the Granulation Blend was charged into the Huttlin Fluid Bed Coating unit and the HPMC Solution from Part A was sprayed at 30-33 g/min.
2. After 350 g of the HPMC solution was sprayed a sample was tested for Loss on Drying (LOD). The Loss on Drying was 3-4% w/w.
3. After 400 g of the HPMC solution was sprayed, 100 g of the Layering Powder was added through the side charging port over 3-4 minutes, while the HPMC solution was sprayed at 30-33 g/min.
4. Powder layering was stopped for 3-4 minutes while the HPMC solution continued spraying at 30-33 g/min.
5. An additional 100 g of powder was added over 3-4 minutes, while the HPMC solution was sprayed at 30-33 g/min.
6. The process of Steps 4-5 was repeated 4 times to layer a total of 410 g of layering powder.
7. Samples were collected at the end of every powder addition and tested for LOD. The LOD was 6-8% weight/weight.
8. The spray rate was reduced to 20-25 g/minute and the bed temperature was raised to convert the granulation process into a coating process.
9. The final seal coat was applied with approximately 150 g of HPMC solution. The LOD of samples at this step were 2-4% weight/weight.
10. The granules were dried at low air volume for 3-5 minutes to a final LOD of less than 1.0% weight/weight.
11. Processing parameters are shown in Table 3.

TABLE 1
Granulation Blend Formula (Similar for Trials 1, 2 and 3)

| | Commercial | | |
|---|---|---|---|
| Ingredient | Supplier | Function | % w/w |
| Famotidine | Gedeon Richter | Active Pharmaceutical | 14.6% |
| | | Ingredient (API) | |
| Lactose Monohydrate | Kerry | Filler | 78.3% |
| Impalpable | | | |
| Syloid 244 FP | WR Grace | Flow aid | 0.5% |
| HPMC E5 LV | Ashland | Binder | 6.6% |
| Water * | | For binder solution | — |

TABLE 2a
Powder Layering Formula for trials 1 and 2

| | | | Granulation + Layering (% w/w) | | |
|---|---|---|---|---|---|
| | Commercial | | Granu- | | Final |
| Component | Supplier | Function | lation | Layering | Particle |
| Lactose | Kerry | Filler | 78.3% | 97.5% | 81.5% |
| Impalpable | | | | | |
| Syloid | WR Grace | Flow aid | 0.5% | 1.0% | 0.6% |
| 244 FP | | | | | |
| HPMC | Ashland | Binder | 6.6% | 1.5% | 5.7% |
| E5 LV | | | | | |

TABLE 2b
Powder Layering Formula for trial 3

| | | | Granulation + Layering (% w/w) | | |
|---|---|---|---|---|---|
| | Commercial | | Granu- | | Final |
| Component | Supplier | Function | lation | Layering | Particle |
| Micro- | FMC | Filler | — | 94.5% | 15.6% |
| crystalline | | | | | |
| Cellulose | | | | | |
| PH 105 | | | | | |
| Lactose | Kerry | Filler | 78.3% | — | 65.4% |
| Impalpable | | | | | |
| Syloid | WR Grace | Flow aid | 0.5% | 1.0% | 0.6% |
| 244 FP | | | | | |
| HPMC | Ashland | Binder | 6.6% | 4.5% | 6.2% |
| E5 LV | | | | | |

TABLE 3
Powder Layering process parameters (Similar for trials 1, 2 and 3)

| Process Step | Process Parameter | Range | |
|---|---|---|---|
| Spray 1, wetting | Inlet Air Temp | 65° | C. |
| | Process Air Flow | 75-100 | m³/hr |
| | Product Temp | 24-26° | C. |
| | Outlet Air Humidity | 16-17 | g/kg |
| | Time | 10-12 min | |
| | | (~30% solution) | |
| Spray 1, layering* | Inlet Air Temp | 65° | C. |
| | Process Air Flow | 75-100 | m³/hr |
| | Product Temp | 24-26° | C. |
| | Time | 20 min | |
| | | (60% solution) | |
| | Outlet Air Humidity | 15-17 | g/kg |
| Spray 2 (Addition of | Inlet Air Temp | 65-69° | C. |
| Second Binder and Seal | Process Air Flow | 100-120 | m³/hr |
| coat) | Product Temp | 30-35° | C. |
| | Outlet Air Humidity | 10-13 | g/kg |
| | Time | 4-7 | min |

*4 powder additions of 100-150 g each were added while spraying the Hypromellose solution.

### Example 2: Tablet Compression

1. The materials in Table 4 were weighed out and manually blended end over end in a plastic bag.
2. 1830 mg tablets were compressed using an Elizabeth tablet press, using 2 punches at 10 RPM, with a precompression force of 2.3 kilonewtons and a main compression force of 22 kilonewtons.
3. Punches were 11/16″ round tooling with a concave center.
4. The final thickness was approximately 5.75 mm and the ejection force was about 400 Newtons
5. Friability of a 6 g sample was less than 1%.
6. The Tablet formula is shown in Table 4 and the compression parameters are shown in Table 5.

TABLE 4
Tablet Formula

| Ingredient | Supplier | g/batch | mg/tablet | % w/w |
|---|---|---|---|---|
| Dextrose Monohydrate | Dominos | 915.79 | 1046 | 57.24 |
| Calcium Carbonate/ | IVC | 562.08 | 642 | 35.13 |
| Magnesium Hydroxide | | | | |
| Granulation | | | | |
| Prosweet Powder # 694 | Virginia Dare | 13.13 | 15 | 0.82 |
| Sucralose Powder NF | Tate & Lyle | 2.19 | 2.5 | 0.14 |
| Crospovidone NF | Ashland | 26.27 | 30 | 1.64 |
| Magnesium Stearate | Mallinckrodt | 8.76 | 10 | 0.55 |
| Famotidine Layered | N/A | 71.79 | 82 | 4.49 |
| Particles (from table | | | | |
| 2a for trials 1 and 2; | | | | |
| from table 2b for trial 3) | | | | |

Note:
Besides Famotidine Layered particles, tablet formulas are similar for each trial.

TABLE 5
Compression Parameters

| | | | | | | Main | | |
|---|---|---|---|---|---|---|---|---|
| | Avg. | | Avg. | | | Com- | Pre- | |
| Trial | Tablet | Target | Thick- | | Hard- | pression | Comp | Ejection |
| # | Weight | Weight | ness | Friability | ness | force | force | Force |
| Trial 1 | 1830.1 mg | 1826.1 mg | 5.74 mm | 0.107% | 8.1 Kp | 22.3 kN | 2.3 kN | 401 N |
| Trial 2 | 1827 mg | 1826 mg | 5.71 mm | 0.234% | 7.9 kp | 22.1 kN | 2.1 kN | 396 N |
| Trial 3 | 1831 mg | 1826 mg | 5.74 mm | 0.211% | 8.2 kp | 22.1 kN | 2.1 kN | 399 N |

kN: Kilonewtons
N: Newtons

### Example 3: Stability Evaluation

The Tablets from Example 2, trial 1, were tested for chemical degradation stability using the conditions in Table 6. 50 tablets were placed into 115 mL HDPE bottles and manually sealed with a lid (not heat sealed). Additional tablets and particles were placed into open dishes for direct exposure to the stability environment.

TABLE 6
Stability Condition and Pull (trial 1)
Stability Condition/Sample Pull

| 40° C., 75% RH | | 25° C., 60% RH |
|---|---|---|
| Open Dish | Closed Container | Closed Container |
| 1 Week, 2 Weeks, +1 | 1 Months, 6 Months, +1 | 6 Months, +1 |

### Example 4: Dissolution Data on Stability (Tablets from Example 2, Trial 1)

Dissolution was analyzed at initial timepoint and in open dish conditions us 0.1M Acetate buffer at pH 4.5 using a reciprocating cylinder set up where instead of a paddle or a basket rotating along its vertical axis, the cylinder with tablets in it is dipped in a dissolution vessel at a predetermined rate (30 dips per min). 900 mL of dissolution media is introduced to each of the six 1 L dissolution vessel and equilibrated at 37ºC. 1 tablet (Example 2, trial 1) is placed in each of the 6 reciprocating cylinder equipped with 20 mesh (840 μm) top screen, 40 mesh (405 μm) bottom screen. Test is started with 30 dips per minute without pause, after 3 min, 10 min and 30 min withdraw about 5 mL of Dissolution Media from a zone midway between the surface of the Dissolution Media and the bottom of each vessel using stainless steel cannulas to syringes fitted with 10-20 μm filters. The volume of the media removed as sample is replenished with fresh dissolution media after each sampling.

The dissolution testing was analyzed versus a dissolution working standard prepared at 0.0112 mg/mL, using an HPLC with the following parameters:
Column: Inertsil ODS-2, 150 mm length×4.6 mm ID, 5 μm particle size
Mobile Phase: Gradient method of 0.1 M Sodium Acetate, pH 6.0: Acetonitrile (over 18 minutes)
Flow rate: 1.6 mL/min
Injection volume: 20 μL
Column temperature: 40° ° C.
UV Detector at 270 nm
Suggested Run Time: 18 min

TABLE 7
Dissolution on Stability (Tablets from Example 2, trial 1)

| | Compressed Tablets containing | | |
|---|---|---|---|
| | Lactose Powder Coated Particle | | |
| | (Timepoint, % released at 30 minutes) | | |
| Time point | 3 min | 10 min | 30 min |
| Initial | 91 | 101 | 102 |
| 1 week Open dish | 91 | 99 | 100 |
| 2 week Open dish | 88 | 99 | 100 |

Note: target was a total of 10 mg Famotidine per tablet, but a typical acceptable range may vary from 95% to 105%. Thus a 102% dissolution is not surprising, it simply due to Famotidine dosage variation.

### Example 5: Famotidine Chemical Evaluation on Stability

Tablets from Example 2 (trials 1, 2 and 3) and powder coated particles from Example 1 (trials 1, 2 and 3) were evaluated for stability, 2 sets of conditions were used:
1) at room temperature (RT, 25° C.) and 60% relative humility (RH),
2) at 40° C. and 75% RH.
   Data were collected for 6 months and 12 months (trial 1) or 9 months (trials 2 and 3).

Method: The famotidine assay were analyzed versus an assay working standard famotidine solution prepared at 80 μg/mL and famotidine impurity assay were analyzed versus a degradation products standard solution prepared at 1.0% FAM (0.8 μg/mL), using an HPLC with the following parameters:
Column: Advanced Chromatography Technologies (ACE) C8, 3 μm (150 mm×4.6 mm I.D.), ACE-112-1546 or EXL-112-1546U.
Mobile Phase: Gradient method of 40 mM KPF6 in 10 mM Sodium Phosphate Buffer:Acetonitrile (98:2 to 30:70, ratio expressed in volume, gradient over 26 minutes)
Flow rate: 1.0 mL/min
Injection volume: 15 L
UV Detector at 278 nm
Flow rate: 1.0 mL/min
Injection volume: 15 μL
Column temperature: 35° C.
UV Detector at 278 nm
Suggested Run Time: 26 min

Multiple degradants were examined on stability as shown in Tables 8 and 9.

TABLE 8
Famotidine Chemical Stability (trial 1), values are expressed in % of degradant
as a percentage of the total dose of famotidine. The following degradants are monitored
as a result of the chemical degradation of famotidine in various conditions including
hydrolysis (FAM-A1 and FAM-A3) and oxidative degradation (FAM-A6).

| | Trial 1 | | | | |
|---|---|---|---|---|---|
| | | 6-Mo | 12-Mo | 12-Mo | 6-Mo |
| | T = 0 | Tablets | Tablets | Particles | Particles |
| | Tablets | 25° C./60% | 25° C./60% | 25° C./60% | 40° C./75% |
| Peak Name | (%) | (%) | (%) | (%) | (%) |
| FAM | 98 | 100.8 | 100.9 | 100.3 | 100.5 |
| FAM-A1 | | ND | ND | 0.07 | ND |
| (hydrolysis) | | | | | |
| FAM-A3 | 0.12 | 0.12 | 0.11 | 0.11 | 0.20 |
| (hydrolysis-base) | | | | | |
| FAM-A6 (oxidative | 0.66 | 0.56 | 0.45 | 0.04 | ND |
| degradation) | | | | | |
| FAM-C1 | ND | ND | ND | ND | ND |
| FAM-DEXT1 | ND | ND | ND | ND | ND |
| FAM-DEXT2 | ND | ND | ND | ND | ND |
| FAM-DEXT3 | ND | ND | ND | ND | ND |
| FAM-ETOH | ND | ND | ND | ND | ND |
| Total | 0.8 | 0.986167 | 0.7 | 0.22 | 0.504187 |
| Degradants | | | | | |

TABLE 9
Famotidine Chemical Stability (trial 2 and 3), values are expressed
in % of degradant as a percentage of the total dose of famotidine

| | Trial 2 | | Trial 3 | |
|---|---|---|---|---|
| | 9 M Tablets | 9 M Particles | 9 M Tablets | 9 M Particles |
| | 25° C./60% | 25° C./60% | 25° C./60% | 25° C./60% |
| Peak Name | (%) | (%) | (%) | (%) |
| FAM | 98.2 | 100.4 | 96.5 | 98.3 |
| FAM-A1 | ND | 0.07 | ND | 0.07 |
| (hydrolysis) | | | | |
| FAM-A3 | 0.13 | 0.10 | 0.10 | 0.09 |
| (hydrolysis- | | | | |
| base) | | | | |
| FAM-A6 | 0.65 | ND | 0.83 | 0.07 |
| (oxidative | | | | |
| degradation) | | | | |
| FAM-C1 | ND | ND | ND | ND |
| FAM-DEXT1 | ND | ND | ND | ND |
| FAM-DEXT2 | ND | ND | ND | ND |
| FAM-DEXT3 | ND | ND | ND | ND |
| FAM-ETOH | ND | ND | ND | ND |
| Total | 0.94 | 0.17 | 1.06 | 0.23 |
| Degradants | | | | |

ND: None Detected

Note: target was a total of 10 mg Famotidine per tablet, but a typical acceptable range may vary from 95% to 105%. Moreover, factors like relative response factor, quantitation standards and its response factor and how the API degrades and combines with other formulation components would dictate % impurity or % degradant. Therefore adding % impurity to Assay may not add up to a 100%. It is desirable to have the total % degradant level at less than 2.0%.

### Example 6: Particle Size Analysis

The particle size distribution of the layered famotidine granules from Example 2 trial 1 was carried out using a sonic sifter. Approximately 7-8 g of powder was accurately weighed out and sifted in a sonic sifter using the following mesh screens, stacked in order.
1. Mesh 20
2. Mesh 50
3. Mesh 60
4. Mesh 80
5. Mesh 100
6. Pan.

After sifting, the difference between the tare weight and final weight was calculated to determine the amount of powder that was retained on each screen. For the example selected above.

TABLE 10
Percent Retained

| 20 | 20 + 50 | 20 + 50 + 60 | 20 + 50 + 60 + 80 | 100 + Pan |
|---|---|---|---|---|
| 0% | 21% | 38% | 68% | 32% |

### Example 7: Particle Friability

Approximately 100 g of Layered Famotidine Granules from example 2 trial 1 were weighed out and put in a 500 g plastic jar. The jar was put in a Turbula mixer simulating a blending process. Particle size measurement is repeated after 10 minutes of blending.

TABLE 11
Percent Retained

| 20 | 20 + 50 | 20 + 50 + 60 | 20 + 50 + 60 + 80 | 100 + Pan |
|---|---|---|---|---|
| 0% | 17% | 48% | 82% | 48% |

### Example 8: Evaluation of Coating Levels and Material Levels as a Design of Experiments

A design of experiments was conducted to evaluate the impact of various total coating levels and amounts of the binder and seal coat (or second binder). A total of 9 batches was produced with layering levels of 20, 25 and 30%; hypromellose levels of 1.5, 2.75 and 4.0 percent and seal coating levels of 0.8, 1.2 and 1.6 percent. Seal coat layer is the final layer of polymer solution (binder 2 in present case Hypromellose) that is sprayed on the particles to create a polymer film coating, that can provide strength and reduce friability of the coated famotidine particle. The outline of experiments is shown in Table 12. Particle size was analyzed using the method in Example 6 and the particle size data is shown in Table 13.

TABLE 12

| | Layering Level | | |
|---|---|---|---|
| Batch | (weight gain) % | HMPC Level | Seal Coat Level¹ |
| 1 | 20 | 4.0 | 0.8 |
| 2 | 20 | 4.0 | 1.6 |
| 3 | 30 | 4.0 | 0.8 |
| 4 | 30 | 1.5 | 1.6 |
| 5 | 25 | 2.75 | 1.2 |
| 6 | 30 | 1.5 | 0.8 |
| 7 | 20 | 1.5 | 0.8 |
| 8 | 20 | 1.5 | 1.6 |
| 9 | 30 | 4.0 | 1.6 |

¹A final layer or “seal coat” comprising HPMC was added to the particles at the end of the spray process

TABLE 13
Particle Size Data for Design of Experiment batches

| Batch | 20 | 20 + 50 | 20 + 50 + 60 | 20 + 50 + 60 + 80 | 100 + Pan |
|---|---|---|---|---|---|
| 1 | 1.4 | 61.6 | 76.6 | 94.1 | 5.9 |
| 2 | 0.2 | 75.5 | 86.3 | 97.9 | 2.1 |
| 3 | 2.1 | 68.1 | 81.5 | 95.7 | 4.3 |
| 4 | 0.1 | 61.8 | 75.5 | 93.8 | 6.2 |
| 5 | 0.0 | 63.4 | 77.0 | 92.8 | 7.2 |
| 6 | 0.0 | 56.1 | 70.0 | 91.0 | 9.0 |
| 7 | 0.5 | 61.7 | 73.1 | 90.4 | 9.6 |
| 8 | 0.0 | 68.2 | 80.5 | 95.7 | 4.3 |
| 9 | 0.2 | 67.8 | 79.5 | 94.0 | 6.0 |

The design of experiments batches were also tested for degradation products FAM A1, FAM A2, FAM A3, FAM A6 and FAM C1 after storage at 1 month and 40° C./75% relative humidity. In addition, the batches from the design of experiments were blended and compressed into tablets using the formula form Example 2. Tablets comprising batches 5, 7 and 9 were also stored at 3 months and 40° C./75% relative humidity. A summary for all batches both as coated particles and/or compressed tablets in all conditions (described above) for batches 1-9 is shown below:
FAM A1 was at 0.10%
FAM A2 had none detected
FAM A3 at 0.2% or below
FAM A6 at 0.1% or below
FAM C1 had none detected

1. A coated famotidine particle having at least a core and a coating layer, wherein
the particle core comprises from 5% to 20% w/w of famotidine, from 70% to 93% w/w of a first filler and from 2% to 10% w/w of a first binder, and
the coating layer is substantially free from famotidine and comprises a second filler and a second binder,
wherein the first and second filler may be similar or different; and selected from the group consisting of lactose, microcrystalline cellulose, starch, dextrose, mannitol, sorbitol, xylitol, maltitol or combination thereof and
wherein the first and second binder may be similar or different, and selected from the group consisting of hydroxypropylmethylcellulose, hydroxypropylcellulose, methylcellulose, polyvinylpyrrolidone, Sodium Carboxymethylcellulose, Ethyl cellulose, Polyvinyl alcohol-povidone copolymer, pregelatinized starch, or combination thereof.

2. The coated famotidine particle according to claim 1, wherein the first and second filler is first and second binder is lactose and the hydroxypropylmethylcellulose.

3. The coated famotidine particle according to claim 1, wherein the core comprises a flowing agent selected from the group consisting of Silica, colloidal silica, fumed silica, aluminometasilicate, preferably a colloidal silica such as amorphous silicon dioxide.

4. The coated famotidine particle according to claim 1, wherein the coating layer accounts for 10% to 30% w/w of the particle total weight.

5. The coated famotidine particle according to claim 1, wherein said core and coating layer comprise:
from 10% to 15% w/w of Famotidine, preferably from 12% to 14% w/w,
from 3% to 10% w/w of Binders, preferably from 5% to 6.5% w/w,
from 75% to 90% w/w of Fillers, preferably from 80% to 83% w/w,
from 0.1% to 1% w/w of a Flowing agent, preferably from 0.5% to 0.7% w/w,
wherein all % w/w are respective to the coated famotidine particle total weight.

6. The coated famotidine particle according to claim 1, wherein the coating layer has a thickness between 50 μm to 300 μm.

7. The coated famotidine particle according to claim 1, wherein the particle size is between 200 μm to 500 μm.

8. A solid dosage form comprising the coated famotidine particle according to claim 1, wherein the coated famotidine particle represents from 3% to 6% w/w of the solid dosage form total weight.

9. The solid dosage form according to claim 8, comprising in addition to the coated famotidine particle an antacid, wherein the antacid represents from 50% to 60% of the dosage form total weight.

10. The solid dosage form according to claim 8, wherein the antacid is selected from the group consisting of calcium carbonate, sodium bicarbonate, magnesium hydroxide, aluminum oxide, aluminum hydroxide, magnesium oxide, magnesium carbonate, aluminum phosphate, magaldrate, magnesium trisilicate, bismuth salicylate, bismuth subsalicylate or combination thereof.

11. The solid dosage form according to claim 8, wherein the famotine content is from 50 mg to 200 mg.

12. Method for manufacturing a coated famotidine particle as disclosed in claim 1, comprising the following steps:
Spraying a first binder on a mixture of famotidine and a granulation batch of a first filler to obtain wet famotidine particles core,
Spraying a second binder, while mixing said wet famotidine particles core and a layering batch comprising a second filler, to coat the famotidine particle core into a coated famotidine particle,
wherein the first and second filler may be similar or different; and the first and second binder may be similar or different.

13. The method according to claim 12, wherein the first filler and the second filler used respectively in the granulation batch and the layering batch are the same.

14. The method according to claim 12, wherein the first binder and the second binder, respectively sprayed on the famotidine and the granulation batch, and sprayed on the wet famotidine particles core and the layering batch, are the same.

15. Use of a coated famotidine particle according to claim 1, for the manufacture of a solid dosage form for the treatment of a disease or disorder in the gastrointestinal tract such as heartburn, bothersome gas symptoms, indigestion, dyspepsia, impaired digestion, upper abdominal fullness, nausea, belching, upper abdominal pain, gastroesophageal reflux disease (GERD) or gastritis.

16. A coated famotidine particle according to claim 1 for use as a treatment of a disease or disorder in the gastrointestinal tract such as heartburn, bothersome gas symptoms, indigestion, dyspepsia, impaired digestion, upper abdominal fullness, nausea, belching, upper abdominal pain, gastroesophageal reflux disease (GERD) or gastritis.

17. A method of treating a disease or disorder in the gastrointestinal tract by use of the coated famotidine particle according to claim 1.

18. A solid dosage form according to claim 8 for use as a treatment of a disease or disorder in the gastrointestinal tract such as heartburn, bothersome gas symptoms, indigestion, dyspepsia, impaired digestion, upper abdominal fullness, nausea, belching, upper abdominal pain, gastroesophageal reflux disease (GERD) or gastritis.

19. A method of treating a disease or disorder in the gastrointestinal tract by use of the solid dosage form according to claim 8.
